(19) United States
(12) Patent Application Publication
(10) Pub. No.: US 2025/0073707 A1
(43) Pub. Date: Mar. 6, 2025

(54) Microfluidic Device and Method for Using a Microfluidic Device

(71) Applicant: Robert Bosch GmbH, Stuttgart (DE)

(72) Inventor: Daniel Sebastian Podbiel, Rutesheim (DE)

(21) Appl. No.: 18/729,644

(22) PCT Filed: Nov. 9, 2022
(86) PCT No.: PCT/EP2022/081199

§ 371 Date: Jul. 17, 2024

(30) Foreign Application Priority Data
Jan. 21, 2022 (DE) 10 2022 200 662.3

(51) Int. Cl. B01L 3/00; C12Q 1/6844

(52) U.S. Cl. CPC B01L 3/502715; C12Q 1/6844; B01L 2200/0684; B01L 2200/16; B01L 2300/087; B01L 2400/0487; B01L 2400/0622

(57) ABSTRACT

A microfluidic device for processing a sample is disclosed. The device includes an amplification functional module having a microfluidic channel for guiding a fluid, a dry reagent pre-storage chamber connected to the channel for pre-storing a dry reagent and a ventilation channel connected to the dry reagent pre-storage chamber for connecting the dry reagent pre-storage chamber to a ventilation opening. The ventilation channel is designed so as to be fluidically separate from the microfluidic channel.

### PRIOR ART

The invention is based on a microfluidic device and a method according to the generic type of the independent claims.

Microfluidic analysis systems (so-called lab-on-chips (LoC for short)) enable automated, reliable, fast, compact and cost-effective processing of patient samples for medical diagnostics. By combining a variety of operations for the controlled manipulation of fluids, complex molecular diagnostic test sequences are performed in a microfluidic device, which is also referred to as a lab-on-chip cartridge. Processing of the lab-on-chip cartridge and analysis of the patient sample can be done in an analyzer. Various types of microfluidic analysis systems are known in the prior art, which can also be referred to as lab-on-chip platforms or lab-on-chip systems. Such lab-on-chip platforms can take various technological approaches for providing the microfluidic operations. For example, centrifugal-based lab-on-chip systems make use of the centrifugal, Coriolis, and Euler forces that occur in a lab-on-chip cartridge placed in controlled rotation. Another class of lab-on-chip platforms are the pressure-based systems, which, by applying at least two pressure levels to a microfluidic cartridge, achieve controlled fluid transport in the cartridge.

### DISCLOSURE OF THE INVENTION

Against this background, the approach presented herein provides a microfluidic device and a method for using a microfluidic device according to the independent claims. Advantageous embodiments of and improvements to the device specified in the independent claim are made possible by the measures presented in the dependent claims.

The approach presented here can be used to provide an improved microfluidic device with active microfluidic elements, in which an amplification reaction can be performed particularly advantageously. By implementing the device presented here with a separation between a microfluidic channel leading to a dry reagent pre-storage chamber and a ventilation channel venting the dry reagent pre-storage chamber, the stability of a dry reagent can be optimized and the dissolution of the dry reagent can be performed in a particularly defined and reliable manner. An undesirable ingress of fluid or moisture into the chamber with the pre-stored dry reagent can be prevented in a particularly advantageous manner, in particular by an advantageous design of the microfluidic device.

A microfluidic device for processing a sample is presented, wherein the device comprises an amplification functional module having a microfluidic channel for guiding a fluid, a dry reagent pre-storage chamber connected to the channel for pre-storing a dry reagent and a ventilation channel connected to the dry reagent pre-storage chamber for connecting the dry reagent pre-storage chamber to a ventilation opening. The ventilation channel is designed so as to be fluidically separate from the microfluidic channel.

For example, the microfluidic device may be a cartridge for use with a pressure-based system in which controlled fluid transport is possible by applying at least two pressure levels, for example for processing sample material. To this end, the microfluidic cartridge can comprise in particular a plurality of pneumatic microchannels for the defined guiding of a gaseous medium applied with overpressure or negative pressure, for example, air or an inert gas, within the microfluidic cartridge as well as a microfluidic network of fluidic microchannels for guiding fluids within the microfluidic cartridge. Thus, the integration of a flexible membrane into the lab-on-chip cartridge combines several advantages. In this way, a targeted deflection of the membrane into defined cavities or recesses provided for this purpose can be exploited as fluid displacement chambers for the realization of microfluidic valves and pumping chambers in the cartridge in order to displace and process defined fluid volumes. Furthermore, the use of a flexible membrane, which is integrated in a lab-on-chip cartridge, means that the fluids are almost completely contained in the cartridge. Only ventilation channels are required. This can prevent contamination of the environment by the sample or vice versa. In addition, these types of microfluidic lab-on-chip cartridges may be produced cost-effectively from polymers using series production methods such as injection molding, injection stamping, punching or laser transmission welding. Control of the active microfluidic elements can, in particular, be carried out by a processing unit or the analyzer, and via pneumatic microchannels, which can be integrated into the microfluidic device. For this purpose, the microfluidic device may in particular comprise a pneumatic interface to the processing unit. The pneumatic interface may be suitably configured to enable control by the processing unit on one hand, and on the other hand, suitably implemented into the microfluidic device to permit control of all active microfluidic elements in the microfluidic device. For example, a plurality of active microfluidic elements may be controlled via a common pneumatic interface having multiple pneumatic ports, which may therefore also be referred to as a manifold. By shaping of the pneumatic interface as a manifold which may comprise a plurality of pneumatic ports, pneumatic control of the microfluidic device in an external processing unit can be achieved in a particularly simple and compact manner. In addition to such a manifold as a pneumatic interface, for example, the processing unit may also have further interfaces to the microfluidic lab-on-chip cartridge, such as thermal interfaces for tempering fluids in the lab-on-chip cartridge or an optical interface for evaluating a fluorescent signal. An important operation in the context of a molecular diagnostic test procedure, which can be performed in such a lab-on-chip cartridge in a processing unit, is the performance of an amplification reaction in order to amplify nucleic acids in a specific manner. A commonly used amplification method is the polymerase chain reaction. This is based on a repeated, cyclic execution of the steps of denaturing, annealing and elongating, which must be carried out at different temperatures, for example at 95° C., 55° C. and 72° C. Consequently, thermal cycling of the reaction volume is required to perform a polymerase chain reaction. First, a reaction mix is necessary for the performance of an amplification reaction, for example a master mix for a polymerase. For example, the reagents required for this can be provided in the form of a freeze-dried, lyophilized dry reagent, for example a so-called reaction bead. In this case, the reaction mix can be prepared by dissolving the dry reagent with, for example, an elution buffer, which can contain in particular purified sample material. In the device presented here, the required dry reagent may be pre-stored in a pre-storage chamber of the microfluidic device provided for this purpose, which is implemented in the network of the device. The dry reagent pre-storage chamber is connected to the microfluidic channel via which, for example, fluid can be fed to the dry reagent pre-storage chamber. Apart from opening into the dry reagent pre-storage chamber, this microfluidic channel is formed separately from the ventilation channel, which may, for example, open from another side into the dry reagent pre-storage chamber. The ventilation channel forms a connection to a ventilation opening of the device. The ventilation channel is formed as a continuous branchless channel, according to one embodiment. In particular, according to one embodiment, the ventilation channel does not have a branch that opens into the microfluidic channel or can be coupled to the microfluidic channel. By implementing a pure ventilation channel at the outlet of the dry reagent pre-storage chamber, which is not wetted with fluid prior to dissolving the dry reagent, the dry reagent may be dissolved in a particularly defined and reliable manner, since no displacement of fluid from the ventilation channel is required in particular for venting the dry reagent pre-storage chamber during filling of the chamber with fluid.

According to one embodiment, the ventilation channel may comprise a fluid retention reservoir for receiving fluid introduced into the dry reagent pre-storage chamber. For example, such a reservoir, which may also be referred to as a fluidic decoupling reservoir, may be arranged near the ventilation opening and configured to receive an excess amount of fluid introduced into the dry reagent pre-storage chamber without allowing it to exit the microfluidic device via the ventilation opening of the ventilation channel. Due to the extensive decoupling of the ventilation channel from the microfluidic network, undesirable intrusion of fluid via the ventilation channel into the dry reagent can may be ruled out by design. Furthermore, the connection of a fluid retention reservoir to the ventilation channel can advantageously prevent undesirable leakage of fluid from the microfluidic device via the ventilation opening of the ventilation channel.

According to a further embodiment, the microfluidic channel may comprise at least two microfluidic pumping chambers connected in series for pumping and additionally or alternatively for temporarily receiving the fluid. For example, the amplification functional module of the device may have a series of two or three PCR pumping chambers to form a PCR strand. The microfluidic pumping chambers may be configured to displace fluids from designated areas of fluid-conducting structures of the device as well as to temporarily receive defined fluid volumes. A pumping chamber, which can be used in particular for the defined storage and measurement of fluids, can for example have a predetermined displacement volume, for example 20 μl, which substantially corresponds to the fluid volume to be processed, or at least a significant fraction thereof. In the precise calculation of the displacement volume of a pumping chamber to process a predetermined fluid, the channel and valve volumes should also be included. In particular, a significant part or the (almost) total volume of a fluid to be processed in a step of a microfluidic process may be received by the pumping chambers, for example. In order to, for example, induce pumping at a flow rate that is as constant and non-variable as possible, peristaltic pumping through peristaltic actuation of at least three homogeneous active microfluidic elements may work well, wherein the at least three active microfluidic elements may have a similar volume and, particularly, nearly the same volume. Peristaltic pumping with three similar, active microfluidic elements can be achieved independently of their (identical) displacement volume, i.e. in particular by the use of microfluidic valves, which can have a small displacement volume, or by the use of microfluidic pumping chambers, which can, in particular, have a larger displacement volume. Advantageously, an analysis of a sample dissolved in a fluid, for example, can be optimized by temporarily receiving a fluid in at least one of the pumping chambers.

According to another embodiment, the channel may comprise a first isolating valve for separating the amplification functional module from a microfluidic network of the device, and additionally or alternatively, a second isolating valve for separating two sub-areas of the channel. For example, a microfluidic valve can be realized by separating two fluid-conducting structures by an in particular pneumatically caused deflection of the membrane into a partial volume of the fluid-conducting structures provided for this purpose and particularly advantageously shaped. The first isolating valve and the second isolating valve may, for example, be arranged to enclose the pumping chambers, in order to advantageously temporarily separate both the entire amplification functional module and the sub-area of the pumping chambers within the module from adjacent areas of the device and to prevent further fluid supply or discharge. Furthermore, for example, a microfluidic pumping chamber can be advantageously used in combination with two microfluidic valves enclosing the pumping chamber in order to realize a pump unit, which allows as great a flow rate as possible in the microfluidic device in as compact a space as possible. This can be achieved in particular by the formation of the pump unit from a pumping chamber with a large displacement volume, which is used for pumping, that is to say for the directed displacement of fluids, and two valves with a small displacement volume, which can only be used for defining and establishing the pumping direction by a suitable actuation scheme. Overall, such a pumping unit can advantageously be characterized by a large pumping volume per pumping step, as well as by a small space requirement for the realization of the pumping unit and a pulsatile, that is to say temporally changeable, flow rate profile.

In addition, the first isolating valve may be configured as a double valve. For example, the first isolating valve, which may also be referred to as the functional module isolating valve, may be realized by a series arrangement of two microfluidic valves connected in the same way on a microfluidic supply channel to the amplification functional module.

Advantageously, it can therefore have a particularly large sealing surface compared to the other microfluidic valves. Moreover, by realizing only one separable fluidic feed channel to the PCR strand as an interface of the amplification functional module with the dry reagent pre-storage chamber and the PCR strand to the fluidic network of the microfluidic device, a particularly good shielding of the dry reagent pre-storage chamber and thus the pre-stored dry reagent can be achieved. For example, the risk of undesirable contact of the dry reagent with a fluid or with gaseous molecules of a vaporized fluid, for example water vapor, may be reduced. In the context of a fully automated molecular diagnostic test procedure, for example, the purification of a sample fluid may advantageously be carried out first in a purification functional module, which may form a separate part of the microfluidic network of the microfluidic device and may be isolated from the amplification functional module. In particular, only the above-mentioned microfluidic feed channel of the microfluidic device may advantageously serve as the only connection between the purification functional module and the amplification functional module. In this way, contamination of the amplification functional module, and in particular of the dry reagent pre-stored in the dry reagent pre-storage chamber, for example, by buffer solutions used in connection with sample purification, can be prevented by design.

According to another embodiment, the channel may comprise a fluid flow control valve for controlling a flow rate of the fluid in the channel. For example, the fluid flow control valve may be arranged at the microfluidic channel between the PCR strand formed by, for example, pumping chambers and the dry reagent pre-storage chamber. This has the advantage that the flow rate can be reduced in a fluid transfer between the PCR strand and the dry reagent pre-storage chamber, in particular in a fluid transfer from the PCR strand to the dry reagent pre-storage chamber. In this way, the dry reagent may be brought into contact with the fluid in a particularly controlled and defined manner in order to bring the dry reagent reliably into solution. For example, the reduction of the flow rate during a fluid transfer between the PCR strand and the dry reagent pre-storage chamber may be achieved by temporarily and/or partially closing the fluid flow control valve, wherein the fluid flow control valve may be used in combination with another microfluidic valve, for example the second isolation valve of the PCR strand, in order to reduce the flow rate.

According to a further embodiment, the ventilation channel may comprise a further isolating valve and the dry reagent pre-storage chamber may be pneumatically actuated, such that pneumatic actuation of the dry reagent pre-storage chamber can transport fluid from it into the PCR strand, wherein closing the further isolating valve prevents fluid from entering the part of the ventilation channel located downstream of the further isolating valve during the pumping step. The further isolating valve is in particular arranged downstream of the dry reagent pre-storage chamber on the side of the ventilation channel.

According to a further embodiment, the dry reagent pre-storage chamber may be lens-shaped, wherein a dimension of the dry reagent pre-storage chamber may be in a vertical direction greater than in a horizontal direction, and wherein a force component of the earth's gravitational field may act along the vertical direction in the operational state of the device. For example, both the dry reagent pre-storage chamber and the pumping chambers may each have a lens-shaped design, wherein the longitudinal axes of the chambers may be aligned approximately, i.e. for example at an acute angle of less than 30°, to the aforementioned vertical direction. In this way, gas bubbles may accumulate advantageously in the upper part of the chambers due to the buoyancy force acting on them in the earth's gravitational field.

According to a further embodiment, the microfluidic channel may be U-shaped. This may advantageously enable a particularly compact shape of the device. All micro-fluidic elements integrated in the amplification module can, for example, be arranged in a row along the channel. For example, by realizing only exactly one microfluidic feed channel to the amplification functional module and the linear arrangement of the microfluidic elements, advantageous purging of the PCR strand in particular can be achieved, wherein purging of further connections to avoid contamination to the microfluidic network may be unnecessary. In particular, the channel volume to be purged may be particularly small and may be performed by pumping a fluid volume back and forth in the pumping chambers of the PCR strand in an efficient manner with a low consumption of purging solution to be used.

According to a further embodiment, the pumping chambers and the dry reagent pre-storage chamber may be arranged on opposite longitudinal sides of the U-shaped channel. For example, in the operational state of the device, the channel may be aligned with the earth's gravitational field such that a force component of the earth's gravitational field may act along the microfluidic elements arranged vertically along the U-shaped microfluidic channel. This has the advantage that gravity-dependent analysis processes in particular may be optimized within the amplification functional module.

According to a further embodiment, the device may comprise an airtight package and a desiccant storable in the dry reagent pre-storage chamber and/or the airtight package. The airtight package may advantageously allow long-term stable storage of the device, for example in an airtight pouch with a desiccant. Additionally or alternatively, the valves of the microfluidic device may be in an embossed state. In this way, moisture that may affect the pre-stored dry reagent is advantageously prevented when the microfluidic device is stored.

In addition, a method of using a variant of the microfluidic device that was previously presented is presented. The method comprises a step of introducing a fluid into the microfluidic channel of the amplification functional module and a step of dissolving a dry reagent pre-stored in the dry reagent pre-storage chamber, wherein the dry reagent pre-storage chamber can be vented and in particular is vented by means of the ventilation channel formed separately from the microfluidic channel. In addition, the method comprises a step of transferring a reaction mix prepared using the dry reagent by means of the fluid within the amplification functional module in order to perform an amplification reaction, particularly in the PCR strand. Such a method advantageously enables defined and reliable processing of a fluid in the microfluidic device.

According to one embodiment, the method may comprise a step of tempering the reaction mix to perform the amplification reaction. For example, a polymerase chain reaction may be performed as an amplification method. This is based on a repeated, cyclic execution of the steps of denaturing, annealing and elongating, which must be carried out at different temperatures, for example at 95° C., 55° C. and 72° C. Consequently, thermal cycling of the reaction volume is required to perform a polymerase chain reaction. In addition to a tempering of the reaction volume in a stationary chamber, which may for example have a heat exchange interface, the abovementioned system also offers the advantage of tempering the reaction fluid by microfluidically pumping the fluid volume back and forth between different microfluidic pumping chambers, the walls of which may have different temperatures. In this way, a particularly rapid thermal cycling of a fluid volume in the device can be achieved, wherein even a low thermal conductivity (such as that of polymers) of the device may be sufficient.

According to a further embodiment, the method may comprise a step of pre-wetting the microfluidic channel with a fluid. The PCR strand can, for example, be wetted with a fluid, in particular with an elution buffer. In so doing, the fluid may be introduced into the PCR strand via the fluidic feed channel and discharged from the PCR strand via the same fluidic feed channel together with a gas, such as air, initially enclosed in the microfluidic channel of the PCR strand. Advantageously, no wetting of the dry reagent in the dry reagent pre-storage chamber is performed with such a procedure.

According to a further embodiment, the method may comprise a step of purging the microfluidic channel with a fluid, wherein the step of purging may be after or during the step of dissolving and prior to the step of transferring. For example, in the step of purging, the PCR strand may be purged with a fluid, in particular with an elution buffer, which may occur after or during the step of dissolving a pre-stored dry reagent, such as a reaction bead, in the dry reagent pre-storage chamber and prior to the step of transferring a reaction mix to the PCR strand. The fluid for purging the PCR strand may be introduced and discharged via exactly one fluidic feed channel.

For example, this method can be implemented in software or hardware, or in a mixed form of software and hardware, for example in a controller.

The approach presented here also creates a control device, which is configured to carry out, control or implement the steps of a variant of the method presented here in corresponding devices. The object of the present invention can also be achieved quickly and efficiently by means of this embodiment variant of the invention in the form of a control device.

For this purpose, the control device can comprise at least one computing unit for processing signals or data, at least one memory unit for storing signals or data, at least one interface to a sensor or an actuator for reading in sensor signals from the sensor or for emitting control signals to the actuator, and/or at least one communication interface for reading in or emitting data embedded in a communication protocol. The computing unit may, for example, be a signal processor, a microcontroller or the like, wherein the memory unit may be a flash memory, an EEPROM, or a magnetic memory unit. The communication interface can be designed to read in or emit data in a wireless and/or wired manner, wherein a communication interface capable of reading in or emitting wired data can read in said data from a corresponding data transmission line, for example electrically or optically, or emit said data to a corresponding data transmission line.

In this context, the term “control device” can be understood to mean an electrical device that processes sensor signals and emits control signals and/or data signals as a function thereof. The control device can comprise an interface, which can be designed as hardware and/or software. For example, given a hardware design, the interfaces can be part of what is referred to as an ASIC system, which contains a wide variety of functions for the control device. However, it is also possible that the interfaces are dedicated integrated circuits or consist at least partly of discrete components. When implemented as software, the interfaces can be software modules present, for example, on a microcontroller alongside other software modules.

In an advantageous configuration, the method is controlled by the control device. The control is in particular carried out via actuators, which enable, for example, the application of different pressure levels to the individual pneumatic connections of the device.

Also advantageous is a computer program product or computer program comprising program code which is stored on a machine-readable carrier or storage medium, e.g., a semi-conductor memory, a hard disk memory, or an optical memory and used in order to perform, implement and/or control the steps of the method according to one of the embodiments described above, in particular if the program product or program is executed on a computer or an device.

Exemplary embodiments of the approach presented herein are shown in the drawings and explained in greater detail in the following description. The figures shows:

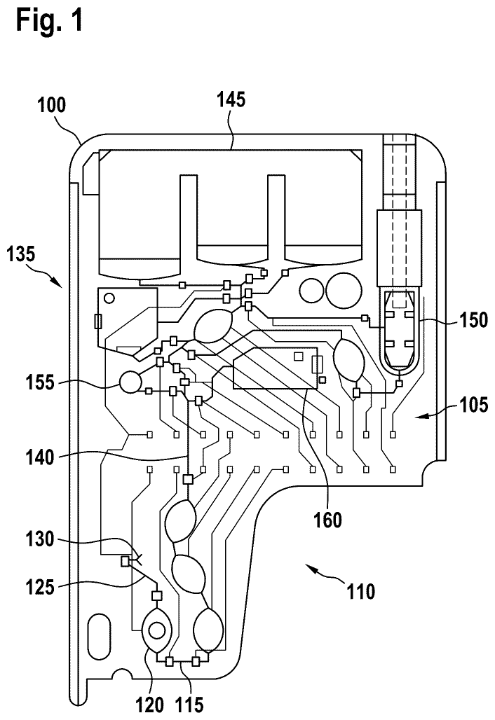

FIG. 1 a schematic top view of a microfluidic device according to an exemplary embodiment;

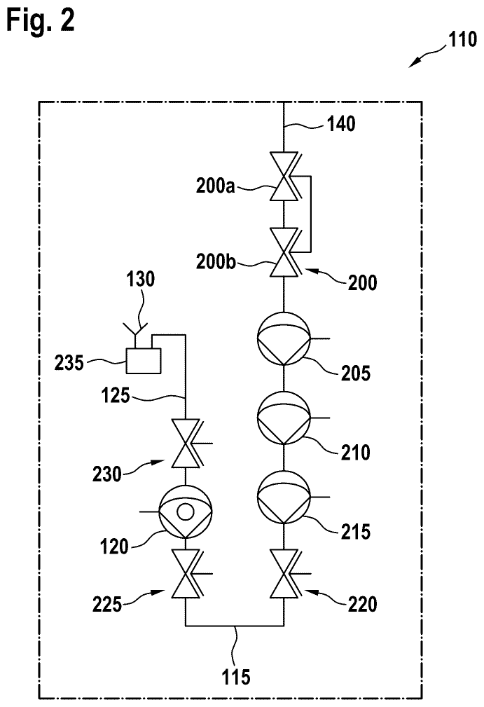

FIG. 2 a schematic illustration of an amplification functional module according to an exemplary embodiment;

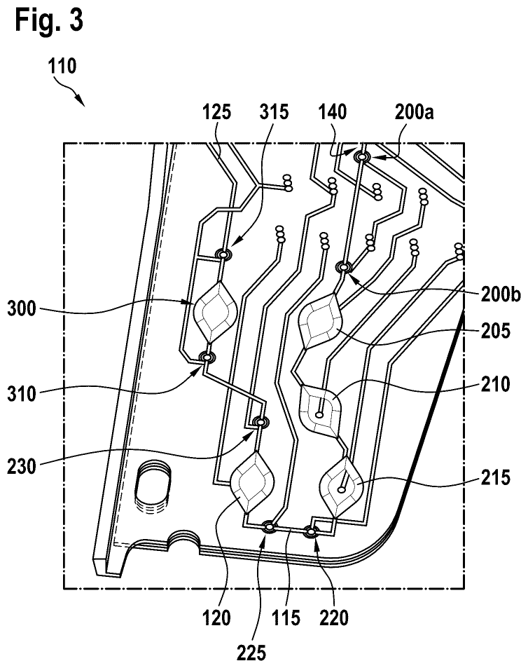

FIG. 3 a perspective view of an exemplary embodiment of an amplification functional module according to an exemplary embodiment;

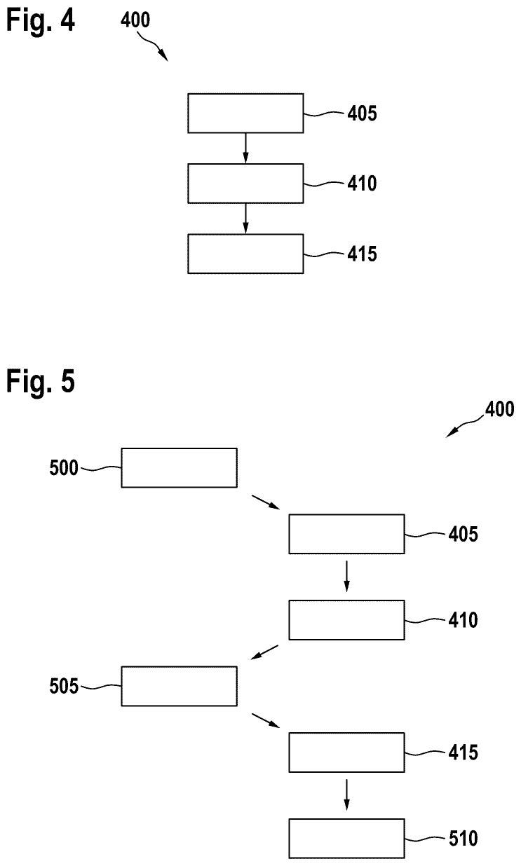

FIG. 4 a flowchart of an exemplary embodiment of a method for using a microfluidic device; and

FIG. 5 a flow chart of an exemplary embodiment of a method for using a microfluidic device.

In the following description of advantageous exemplary embodiments of the present invention, identical or similar reference signs are used for elements shown in the various drawings having a similar function, so a repeated description of these elements has been omitted.

FIG. 1 shows a schematic illustration of a microfluidic device 100 according to an exemplary embodiment. Merely by way of example, the device has a lateral dimension of 118×78 mm². In an alternative exemplary embodiment, the dimensions may also be 30×30 mm² to 300×300 mm², preferably 50×50 mm² to 200×200 mm². In this exemplary embodiment, the microfluidic device 100, which may also be referred to as a cartridge, is configured with a pressure-based system that achieves controlled fluid transport in the microfluidic cartridge by applying at least two pressure levels to the microfluidic cartridge. To this end, the microfluidic device 100 comprises a plurality of pneumatic microchannels for the defined guiding of a gaseous medium applied with overpressure or negative pressure, for example, air, within the microfluidic cartridge, as well as a microfluidic network 105 of fluidic microchannels for guiding fluids within the microfluidic cartridge. In other words, active microfluidic elements, in particular membrane-based valves and pumping chambers, are used to control or produce fluid transport within such a microfluidic cartridge. The microfluidic flow through a channel can be controlled or a microfluidic transport of fluids can be produced by deflecting a flexible membrane, for example made of thermoplastic elastomers (TPE) such as polyurethane (TPU) or styrene block copolymer (TPS), on a valve saddle or into a pumping chamber.

The device 100 comprises an amplification functional module 110 connected to the network 105 having a microfluidic channel 115 for guiding a fluid, a dry reagent pre-storage chamber 120 connected to the channel 115 for pre-storing a dry reagent and a ventilation channel 125 connected to the dry reagent pre-storage chamber 120 for connecting the dry reagent pre-storage chamber 120 to a ventilation opening 130. Here, the ventilation channel 125 is fluidically separate from the microfluidic channel 115, apart from for the dry reagent pre-storage chamber 120, into which both channels 115, 125 open. According to an exemplary embodiment, the ventilation channel 125 runs directly and continuously between the dry reagent pre-storage chamber 120 and the ventilation opening 130, in particular without branching out to the microfluidic channel 115 or to a microfluidic unit connected to the microfluidic channel 115. According to an exemplary embodiment, the ventilation opening 130 is shaped such that fluid located within the ventilation channel 125 can exit the ventilation channel 125 exclusively via the dry reagent pre-storage chamber 120 or the ventilation opening 130. Conversely, a fluid may enter the ventilation channel 125 exclusively via the dry reagent pre-storage chamber 120 or the ventilation opening 130. Apart from the dry reagent pre-storage chamber 120, the ventilation channel 125 and the microfluidic channel 115 are sealed from each other by fluid-impermeable material of the cartridge according to an exemplary embodiment.

In an exemplary embodiment, the microfluidic device 100 overall comprises a microfluidic network 105 with a purification functional module 135 connected to the amplification functional module 110 via a microfluidic feed channel 140. In an exemplary embodiment, the microfluidic network 105 is composed in particular of an arrangement of active microfluidic elements, such as valves and pumping chambers, which are connected to each other and to further passive elements, i.e. elements that cannot be pneumatically controlled, via a network of microfluidic channels. The further passive elements are, for example, fluid reagent pre-storage chambers 145, at least one (sealable) sample input chamber 150, a filter chamber 155 with integrated filter element, at least one fluid storage chamber 160, and ventilation openings.

A microfluidic valve is only exemplary realized by the separation of two fluid-conducting structures by an in particular pneumatically caused deflection of the membrane into a partial volume of the fluid-conducting structures provided for this purpose and particularly advantageously shaped. A microfluidic pumping chamber is closely related to the valve and is also based on displacement of fluids from designated areas of fluid-conducting structures of the device. With respect to peristaltic fluid transport, a conceptual distinction between “valve” and “pumping chamber” is unnecessary. The conceptual separation is only useful if there is a multifunctional use of the microfluidic elements: A microfluidic element, which, in addition to producing a peristaltic fluid transport, is primarily used in order to control the microfluidic flow within the microfluidic device, is therefore referred to as a microfluidic valve. A microfluidic element, which, in addition to producing a peristaltic fluid transport, is used primarily to generate the microfluidic flow as well as for the interim storage of a significant part of the fluid volume to be processed within the microfluidic device, is therefore in particular referred to as a microfluidic pumping chamber.

Depending on the functionalities used of a microfluidic element, an exemplary advantageous configuration is as follows: A microfluidic valve, and in particular a microfluidic control or isolating valve, that is to say a microfluidic valve used exclusively for controlling the microfluidic flow or for separating fluid-conducting structures (and not for peristaltic fluid transport), therefore, has as little displacement volume as possible in particular, in order to, on the one hand, have as low a displacement volume as possible, which can be flushed in a microfluidic drain, if necessary, and on the other hand, to achieve the most compact possible implementation of the microfluidic device.

Regardless of the fluid transport mechanism and the exact configuration of an active microfluidic element, fluid transport in an exemplary embodiment is achieved within the microfluidic device as described above by deflecting a flexible polymeric membrane into fluid-conducting recesses of a rigid polymeric component, such that controlled displacement of fluids within the microfluidic device can be achieved, particularly by applying different pressure levels to a pneumatic interface of the device. In addition to the active, that is pneumatically controllable, microfluidic elements for producing and controlling the fluid transport in the fluidic network, the device as a whole comprises in particular the passive components described below: The fluid reagent pre-storage chambers 145 are particularly for long-term stable pre-storage and defined releasability of fluid reagents needed to perform a test run in the device. The pre-storage chambers in particular contain, by way of example, all fluid reagents needed to perform a test run. For example, aqueous solutions as a whole can be used, in particular buffer solutions, in particular with components of a sample fluid. In this way, with the exception of the sample, no input of further fluids into the device is necessary to have a fully automated test run performed within the device.

The sample input chamber 150 in this exemplary embodiment is configured to introduce a sample, for example to introduce a flocked swab with a swab sample or a sample fluid, that is a fluid with components of a sample. Here an aqueous solutions with sample material contained therein, in particular with sample material of human origin derived from, for example, bodily fluids, swabs, secretions, sputum, or tissue samples, can be introduced. The targets to be detected in the sample fluid are particularly of medical, clinical, diagnostic or therapeutic relevance and may be bacteria, viruses, certain cells, such as circulating tumor cells, cell-free DNA or other biomarkers. The sample input chamber may be sealed with a lid element, by way of example only, in order to exclude contamination of the environment by the sample or vice versa of the sample by the environment.

Moreover, in this exemplary embodiment, the device 100 comprises a filter chamber 155 with a filter element for extraction of constituents from a sample fluid. Merely as an example, the filter element is a silica fabric suitable for extraction of deoxyribonucleic acids (DNA) or ribonucleic acids (RNA).

The fluid storage chamber 160 is configured in an exemplary embodiment to receive and store fluids, such as portions of a sample fluid, particularly after the extraction of components through the filter element. It is possible, for example, for the fluids to remain in the fluid storage chamber without contamination of the environment by the fluid, even after a test run has already been performed in the cartridge. The latter may in particular be ensured by the implementation of a decoupling reservoir between the fluid storage chamber 160 and the ventilation opening.

The ventilation openings serve to vent the microfluidic system and, in particular, provide pressure compensation within the microfluidic system, for example during a pumping operation of a fluid from one of the fluid reagent pre-storage chambers 145 into the fluid storage chamber 160. In particular, decoupling reservoirs are arranged in the microfluidic network 105 upstream of the ventilation openings, which prevent an undesirable leakage of fluid from the device 100.

The active and passive elements of the device 100 described in this exemplary embodiment are connected via microfluidic channels and thus form the microfluidic network 105, which comprises a suitable topology, i.e. spatial arrangement of the elements constituting the network 105, in order to be able to perform at least one and in particular a plurality of different performed microfluidic test procedures in the network 105.

The specific realization of the device 100 is particularly based on a flexible, microstructured polymer membrane, which has been partially welded to two microstructured polymer components by laser welding, which can also be referred to as laser transmission welding. In one exemplary embodiment, the active microfluidic elements can in particular be pneumatically controlled from a processing unit provided for this purpose via an interface with pneumatic ports, such that fully automated microfluidic processing of the fluids in the polymeric cartridge can be achieved.

In a further exemplary embodiment, the multi-layered device may be achieved by utilizing series manufacturing methods such as injection molding, injection compression, and/or punching. Polymers such as polycarbonate (PC), styrene acrylonitrile copolymer (SAN), polypropylene (PP), polyethylene (PE), cycloolefin copolymer (COP, COC), polymethyl methacrylate (PMMA) may be employed.

FIG. 2 shows a schematic illustration of an amplification functional module 110 according to an exemplary embodiment. The amplification functional module 110 shown here corresponds or is similar to the amplification functional module described in the previous figure and is formed as part of a microfluidic device as described in the previous figure. In other words, FIG. 2 shows a schematic representation of an excerpt of an exemplary embodiment of a microfluidic device with features described in the preceding figure and with an amplification functional module 110.

In the exemplary embodiment shown here, the amplification functional module 110 comprises a microfluidic channel 115 having a series of multiple microfluidic elements. In so doing, the amplification functional module 110 is connected to the microfluidic network of the device via a microfluidic feed channel 140, wherein a first isolating valve 200 arranged on the feed channel 140 is configured to separate the amplification functional module 110 from the network. In this exemplary embodiment, the first isolating valve 200, which may also be referred to as a functional module isolating valve or first PCR strand isolating valve, is realized merely by way of example in the form of a double valve. Accordingly, in this exemplary embodiment, the first isolating valve 200 comprises a valve 200a and a further valve 200b, the simultaneous switching of which enables an increased sealing surface to be achieved.

In the illustration shown here, in the series of microfluidic elements of the amplification functional module 110, the first isolating valve 200 is followed merely by way of example by three pumping chambers 205, 210, 215 for forming a PCR strand, which can be separated from the other microfluidic elements of the amplification functional module 110 by means of a second isolating valve 220, which may also be referred to as a second PCR strand isolating valve. The first and second PCR strand isolating valves enclose the PCR pumping chambers and the elements of the PCR strand are connected to each other via the microfluidic channel 115. Merely by way of example, the first isolating valve 200, the pumping chambers 205, 210, 215 and the second isolating valve 220 are arranged on a longitudinal side of the exemplary U-shaped channel 115.

In this exemplary embodiment, a dry reagent pre-storage chamber 120 is arranged on a side of the U-shaped channel 115 opposite the PCR strand. This is configured to pre-store a dry reagent for performing an amplification reaction and is connected to the PCR strand via the microfluidic channel 115 with a fluid flow control valve 225. On the opposite side of the fluid flow control valve 225, a ventilation channel 125 opens into the dry reagent pre-storage chamber 120, which can be separated by a further isolating valve 230. The ventilation channel 125 is thus directly attached to the dry reagent pre-storage chamber 120 and terminates in a ventilation opening 130. The ventilation channel 125 is characterized in that it neither comprises a connection channel to the microfluidic feed channel 140 nor to the microfluidic channel 115. The only connection to the microfluidic channel 115 is the dry reagent pre-storage chamber 120, which in turn can be separated from the channels 115, 125 by means of the fluid flow control valve 225 and the further separation valve 230. Accordingly, fluid intrusion into the ventilation channel 125 is minimized. In addition, in this exemplary embodiment, a fluid receiving reservoir 235 is attached to the ventilation channel 125 in order to receive any excess fluid in the ventilation channel 125.

In one exemplary embodiment, a displacement volume of a microfluidic pumping chamber 205, 210, 215 of the PCR strand is, for example, 20 μl. Optionally, the displacement volume may be 10 μl to 50 μl, preferably 15 μl to 25 μl. The displacement volume of a microfluidic valve 200a, 200b, 220, 225, 230, on the other hand, is merely by way of example 30 μl to 1 μl, preferably 100 μl to 300 μl.

In an exemplary embodiment, the volume of the dry reagent pre-storage chamber 120 comprises, for example, 25 μl and the volume of the portion of the microfluidic channel 115 between the PCR strand and dry reagent pre-storage chamber comprises, merely by way of example, about 5 μl, such that the total volume of the dry reagent pre-storage chamber 120 and the portion of the microfluidic channel 115 for dissolving the beads is, merely by way of example, only about 30 μl. Optionally, the dry reagent pre-storage chamber 120 may also have a displacement volume of 10 μl to 50 μl, preferably 20 μl to 30 μl.

The stated dimensions and specifications are examples. For the design and functionality of the device 100, the properties of the fluids used and the material and surface properties of the materials used are also important for implementation of the device.

FIG. 3 shows a perspective view of an exemplary embodiment of an amplification functional module 110 according to an exemplary embodiment. The amplification functional module 110 shown here corresponds or is similar to the amplification functional module described in the previous figures. In this exemplary embodiment, the amplification functional module 110 of the microfluidic device comprises a microfluidic channel 115 with a series arrangement of exactly one microfluidic feed channel 140, a functional module isolating valve, which in this exemplary embodiment is in the form of a double valve 200a, 200b, a row of three pumping chambers 205, 210, 215 for forming a PCR strand, a second isolating valve 220, wherein the first and second PCR strand isolating valves enclose the PCR pumping chambers and the elements of the PCR strand are connected to each other via the microfluidic channel 115, and a dry reagent pre-storage chamber 120 having a further isolating valve 230, which is connected to the PCR strand via the microfluidic channel 115 with fluid flow control valve 225. Merely by way of example, in this exemplary embodiment the amplification functional module 110 comprises a further dry reagent pre-storage chamber 300, which is enclosed by two valves 310, 315, as well as a ventilation channel 125, which is attached to the dry reagent pre-storage chambers 120, 300 and terminates in a ventilation opening, characterized in that that the ventilation channel 125 does not have a connection channel to the microfluidic feed channel 140 or the microfluidic channel 115.

The lateral dimensions of the entire device are 118 mm×78 mm in one exemplary embodiment. The PCR chambers have an exemplary volume of about 20 μl each, which can be used as the reaction volume for performing an amplification reaction. The dry reagent pre-storage chamber 120 and the microfluidic channel 115 have volumes of for example 25 μl and 5 μl respectively, corresponding to a total volume of 30 μl in which dissolution of the dry reagent can be performed. In the advantageous exemplary embodiment shown herein, the dry reagent pre-storage chambers 120, 300 and the pumping chambers 205, 210, 215 are in particular lens-shaped, wherein the longitudinal axes of the chambers are aligned approximately, i.e. exemplary at an acute angle of less than 30°, to the stated vertical direction. In the operational state, the device is preferably suitably aligned with the earth's gravitational field, such that gas bubbles that may form when the dry reagent is dissolved can be discharged into the upper part of the dry reagent pre-storage chamber 120, driven by gravity, by the buoyancy force acting on the gas bubbles due to the difference in density from the surrounding fluid, whereby the reaction mix can be transferred to the PCR strand (virtually) free of gas bubbles. The gravitational field for the buoyancy-driven discharge of gas bubbles is, for example, the earth's gravitational field with a gravitational acceleration of approximately 9.81 m/s². The device 100 can in particular be aligned with the field lines of the gravitational field at a predetermined angle or angular range. The alignment is carried out by way of example at an angle of between 0 and 45°, for example at an angle of 30°.

FIG. 4 shows a flow chart of a method 400 for using a microfluidic device according to one exemplary embodiment. The method 400 shown here may be used to use an device as described in the preceding figures.

The method 400 includes a step 405 of introducing a fluid into the microfluidic channel of the amplification functional module. In an exemplary embodiment, a fluid is fed to the amplification functional module via the exactly one microfluidic supply channel. The fluid is, for example, an elution buffer with constituents of a sample fluid that is used to dissolve the dry reagent pre-stored in the dry reagent pre-storage chamber. The fluid is in particular fed to the amplification functional module by aspirating by means of the PCR pumping chambers.

The method 400 further comprises a step 410 of dissolving a pre-stored dry reagent in the dry reagent pre-storage chamber, wherein the dry reagent pre-storage chamber can be vented using the ventilation channel formed separately from the microfluidic channel. In so doing, the dry reagent pre-stored in the dry reagent pre-storage chamber, for example a reaction bead, is dissolved by the fed fluid, so that a reaction mix is prepared. When the fluid is introduced into the dry reagent pre-storage chamber, the chamber is vented, in particular via the ventilation channel configured for this purpose. As the ventilation channel does not have any attachment to the microfluidic network up to the dry reagent pre-storage chamber, an undesirable fluid entry into the dry reagent pre-storage chamber during venting of the chamber is advantageously prevented.

In addition, the method 400 comprises a step 415 of transferring a reaction mix prepared using the dry reagent by means of the fluid within the amplification functional module in order to perform an amplification reaction. Here, the reaction mix is transferred from the dry reagent pre-storage chamber into the PCR strand. Merely by way of example, the lens-shaped configuration of the dry reagent pre-storage chamber and an appropriate alignment of the device to the earth's gravitational field advantageously ensures that gas bubbles produced during the dissolution of the dry reagent accumulate in the upper region of the reaction mix in the dry reagent pre-storage chamber and are not transferred to the PCR strand.

In other words, a reaction mix is provided for performing an amplification reaction, such as a master mix for a polymerase chain reaction. In this exemplary embodiment, the reagents required for this are provided in the form of a freeze-dried, lyophilized dry reagent, for example a so-called reaction bead. In this case, the reaction mix is prepared by dissolving the dry reagent with an elution buffer, which contains in particular purified sample material. Here, the dry reagent is stored in a pre-storage chamber of the microfluidic device provided for this purpose, which is implemented into the network of the device via at least two microfluidic channels, wherein the first microfluidic channel is used to feed fluid to the dry reagent pre-storage chamber and the second microfluidic channel is used to vent the dry reagent pre-storage chamber. The separate design of the two microfluidic channels prevents fluid intrusion in the dry reagent pre-storage chamber in an undesirable manner.

FIG. 5 shows a flow chart of a method 400 for using a microfluidic device according to one exemplary embodiment. The method 400 shown in this case corresponds or is similar to the method described in the preceding FIG. 4, with the difference that it has additional steps.

In this exemplary embodiment, the method 400 comprises a step 500 of pre-wetting the microfluidic channel with a fluid. In this step 500, the PCR strand is pre-wetted with a fluid, in particular with an elution buffer, wherein the fluid is introduced into the PCR strand via the fluidic feed channel and is discharged from the PCR strand via the same fluidic feed channel along with a gas, such as air, initially enclosed in the microfluidic channel of the PCR strand. In this case, the dry reagent is not wetted in the dry reagent pre-storage chamber. In this way, air present in particular in the microfluidic channel of the PCR strand is discharged before a sample fluid is fed in step 405. In this way, the amount of gas bubbles formed when aspirating the sample fluid into the PCR strand is minimized.

In this exemplary embodiment, step 410 of dissolving is also followed by step 505 of purging the microfluidic channel with a fluid. In this step 505, the PCR strand is purged with a fluid, in particular with an elution buffer. In this way, the PCR strand is purified of possible impurities. In another exemplary embodiment, the step of purging may additionally or alternatively occur during the step of dissolving.

In this exemplary embodiment, step 505 of purging is followed by step 415 of transferring. Then, in this exemplary embodiment, a step 510 of tempering the reaction mix to perform the amplification reaction follows. In this step 510 of tempering, the reaction mix in the PCR strand is tempered to perform an amplification reaction. Merely by way of example, cyclic tempering is performed for the performance of a polymerase chain reaction. This is based on a repeated, cyclic execution of the steps of denaturing, annealing and elongating, which must be carried out at different temperatures, for example at 95° C., 55° C. and 72° C. Consequently, thermal cycling of the reaction volume is required to perform a polymerase chain reaction.

In a particularly advantageous exemplary embodiment, in addition to tempering of the reaction volume in a stationary chamber, which may have a heat exchange interface, it is also possible to temper the reaction fluid by microfluidically pumping the fluid volume back and forth between different microfluidic pumping chambers, the walls of which may have different temperatures. In this way, a particularly rapid thermal cycling of a fluid volume in the device can be achieved, wherein even a low thermal conductivity (such as that of polymers) of the device may be sufficient.

In an alternative exemplary embodiment, a constant temperature may be established to cause an isothermal amplification reaction in the reaction mix.

In an advantageous exemplary embodiment, the method may also comprise a step of long-term storage of the device. In the step, the microfluidic device may be pre-stored with a desiccant in an airtight pouch prior to performing the remaining method steps. Additionally or alternatively, the valves of the microfluidic device may be in an embossed state. In this way, if the microfluidic device is stored, moisture may advantageously be avoided from adversely affecting the pre-stored dry reagent.

1. A microfluidic device for processing a sample, comprising an amplification functional module having (i) a microfluidic channel configured to guide a fluid, (ii) a dry reagent pre-storage chamber connected to the channel and configured to pre-store a dry reagent, and (iii) a ventilation channel connected to the dry reagent pre-storage chamber and configured to connect the dry reagent pre-storage chamber to a ventilation opening, wherein the ventilation channel is designed so as to be fluidically separated from the microfluidic channel.

2. The device according to claim 1, wherein the ventilation channel comprises a fluid retention reservoir configured to receive fluid introduced into the dry reagent pre-storage chamber.

3. The device according to claim 1, wherein the microfluidic channel comprises at least two successively switched microfluidic pumping chambers configured to pump and/or temporarily receive the fluid.

4. The device according to claim 1, wherein the microfluidic channel comprises a first isolating valve configured to separate the amplification functional module from a microfluidic network of the device and/or a second isolating valve configured to separate two sub-areas of the channel.

5. The device according to claim 4, wherein the first isolating valve is configured as a double valve.

6. The device according to claim 1, wherein the microfluidic channel comprises a fluid flow control valve configured to control a flow rate of the fluid in the channel.

7. The device according to claim 1, wherein:
the ventilation channel comprises a further isolating valve, and
the dry reagent pre-storage chamber is pneumatically actuatable.

8. The device according to claim 1, wherein;
the dry reagent pre-storage chamber is lens-shaped,
a dimension of the dry reagent pre-storage chamber is greater in a vertical direction than in a horizontal direction, and
a force component of the earth's gravitational field acts along the vertical direction in the operational state of the device.

9. The device according to claim 1, wherein the microfluidic channel is U-shaped.

10. The device according to claim 9, wherein the pumping chambers and the dry reagent pre-storage chamber are arranged on opposite longitudinal sides of the U-shaped channel.

11. The device according to claim 1, further comprising an airtight package and a desiccant present in the airtight package.

12. A method for using a microfluidic device according to claim 1, the method comprising:
introducing a fluid into the microfluidic channel of the amplification functional module of the microfluidic device;
dissolving a pre-stored dry reagent in the dry reagent pre-storage chamber, wherein the dry reagent pre-storage chamber is configured to be vented by way of the ventilation channel formed separately from the microfluidic channel; and
transferring a reaction mixture prepared using the dry reagent by way of the fluid in the amplification functional module to perform an amplification reaction.

13. The method according to claim 12, further comprising tempering the reaction mix to perform the amplification reaction.

14. The method according to claim 12, further comprising pre-wetting the microfluidic channel with a fluid.

15. The method according to claim 12, further comprising purging the microfluidic channel with a fluid, wherein the step of purging occurs after or during the step of dissolving and prior to the step of transferring.
